(19) United States
(12) Patent Application Publication
(10) Pub. No.: US 2004/0022853 A1
(43) Pub. Date: Feb. 5, 2004

(54) Polymer-based, sustained release drug delivery system

(75) Inventors: Paul Ashton, Boston, MA (US); Jianbing Chen, Belmont, MA (US); Hong Guo, Belmont, MA (US)

Correspondence Address: ROPES & GRAY LLP, ONE INTERNATIONAL PLACE, BOSTON, MA, 02110-2624, US

(73) Assignee: Control Delivery Systems, Inc., Watertown, MA (US)

(21) Appl. No.: 10/449,855

(22) Filed: May 30, 2003

Related U.S. Application Data
(63) Continuation-in-part of application No. 10/134,033, filed on Apr. 26, 2002.
(63) Continuation-in-part of application No. PCT/US02/13385, filed on Apr. 26, 2002.
(60) Provisional application No. 60/286,343, filed on Apr. 26, 2001.
(60) Provisional application No. 60/322,428, filed on Sep. 17, 2001.
(60) Provisional application No. 60/372,761, filed on Apr. 15, 2002.

(51) Int. Cl. A61K 9/22

(52) U.S. Cl. 424/468

(57) ABSTRACT

Disclosed is a sustained release system that includes a polymer and a pharmaceutically active agent dispersed in the polymer. The agent is in granular or particulate form, and has a rate of release from the system that is limited primarily by the rate at which the agent dissolves from the granules into the polymer matrix. Advantageously, the polymer is permeable to the agent and is non-release-rate-with limiting with respect to the rate of release of the agent from the polymer.

### RELATED APPLICATIONS

This application claims the benefit of U.S. Application No. 60/286,343, filed Apr. 26, 2001; U.S. Application No. 60/322,428, filed Sep. 17, 2001; U.S. Application No. 60/372,761, filed Apr. 15, 2002; PCT Application No. U.S. Ser. No. 02/13385, filed Apr. 26, 2002, and U.S. application Ser. No. 10/134033, filed Apr. 26, 2002, the specifications of each of which are incorporated by reference herein.

### FIELD OF THE INVENTION

The present invention relates generally to an improved system of delivering drugs. In particular, the present invention relates to a polymer-based, sustained-release drug delivery system and methods of delivering drugs using the same.

### BACKGROUND OF THE INVENTION

The desirability of sustained release has long been recognized in the pharmaceutical field. Many polymer-based systems have been proposed to accomplish the goal of sustained release. These systems generally have relied upon either degradation of the polymer or diffusion through the polymer as a means to control release.

Implantable drug delivery devices offer an attractive alternative to oral, parenteral, suppository, and topical modes of administration. For example, as compared to oral, parenteral and suppository modes of administration, implantable drug delivery permits more localized administration of drug than do other modes of administration. Thus, implantable drug delivery devices are especially desirable where a clinician wishes to elicit a more localized therapeutic pharmaceutical effect. Additionally, the ability of implantable drug delivery devices to deliver the drug directly to the desired site of action permits the clinician to use drugs that are relatively poorly absorbed, or labile in biological fluids, often to great advantage. Implantable drug delivery devices allow achievement of therapeutic doses at the desired site of action, while maintaining low or negligible systemic levels. Thus implantable drug delivery devices are especially attractive in situations where the drugs in question are toxic or have poor clearance characteristics, or both.

As compared to topical modes of administration, implantable drug delivery devices have the advantage that they can be applied subcutaneously. They can be injected or surgically implanted and thereby deliver drug locally and in high concentrations over a protracted period of time. In comparison, topical application of drugs generally is limited to the epidermis, and must be repeated periodically to maintain concentration of the drug in its therapeutically effective range. Delivery by a transdermal route, such as by a transdermal patch, has the disadvantage of delivering drug systemically.

Despite the obvious advantages of implantable drug delivery devices, there are several needs left to be satisfied by implantable devices. For instance, there is a need for a simple drug delivery device that releases drug at a constant rate. Prior art attempts to solve this problem have met with limited success because they were difficult to construct and inconvenient to use.

Other limitations in the field give rise to a need for drug formulations and delivery devices that provide localized administration of a drug. For example, modern surgical methods employ various and numerous devices that are routinely placed within the body and left there for extended periods of time. Such devices include, but are not limited to stents, surgical screws, prosthetic joints, artificial valves, plates, pacemakers, etc. Such devices have proven useful over time; however some problems associated with implanted surgical devices remain. For instance, stents and artificial valves may be associated with restenosis after vascular surgery. It is therefore often necessary to use systemic drugs in conjunction with implantation of surgical devices, which increases the risk of post-operative hemorrhage. Occasionally, surgical implants may be subject to immune response or rejection. Consequently, it is sometimes necessary to abandon surgical implant therapy, or to use immunosuppressant drugs in conjunction with certain surgical implants. The surgical procedure itself may also give rise to complications, such as infection, pain and swelling. In any event, clinicians have typically had to apply combating medications systematically, rather than through localized administration, leading to a variety of problems and conditions.

In an effort to avoid systemic treatment, the use of drugs in rate-controlling bioerodible polymers has been frequently reported. Such systems are designed to release drug as the polymer erodes. Although helpful and compatible with the present invention, this approach may limit the selection of drug and polymer. There is therefore a need for an improved drug delivery device that provides sustained-release drug delivery within a body over a prolonged period of time that provides localized administration of drugs, does not require complicated manufacturing processes, and can be adapted to function with a variety of drugs and polymers.

### SUMMARY OF THE INVENTION

The present invention includes a sustained-release formulation comprising a therapeutically effective amount of at least one agent coated by or dispersed in a polymer matrix, wherein the agent is in granular or particulate form. The agent is released from the formulation as drug from the granules dissolves into or within the matrix, diffuses through the matrix, and is released into the surrounding physiological fluid. The rate of release is limited primarily by the rate of dissolution of the agent from the granules/particles into the matrix; the steps of diffusion through the matrix and dispersion into the surrounding fluid are primarily not release-rate-limiting.

In certain embodiments, the invention includes a sustained-release formulation comprising at least one granule having a therapeutically effective amount of at least one agent, and a polymer matrix coating the at least one agent, wherein the at least one agent has a rate of release from the formulation that is limited primarily by the rate at which the agent dissolves from the at least one granule into the matrix.

In other embodiments, the invention includes a drug delivery device comprising a substrate having a surface, and a sustained-release formulation adhered to the surface, the sustained-release formulation comprising at least one granule having a therapeutically effective amount of at least one agent dispersed in a polymer matrix, wherein the at least one agent has a solubility in the polymer matrix of about 0.01 mg/ml or less.

Other embodiments include a method of providing sustained-release administration of granular drugs by providing a therapeutically effective amount of at least one agent in granular form, forming a sustained-release formulation by combining the at least one agent with a polymer matrix such that the at least one agent remains substantially in granular form, wherein the at least one agent has a solubility in the polymer matrix of about 0.01 mg/ml or less, providing the sustained-release formulation in a pharmaceutically acceptable carrier, and administering the sustained-release formulation to a patient.

Certain embodiments provide a sustained release system comprising a polymer matrix and a therapeutically effective amount of an agent dispersed in the polymer. In certain embodiments, the agent may have a general formula of A—L—B in which: A represents a drug moiety having a therapeutically active form for producing a clinical response in a patient; L represents a covalent linker linking A and B to form a codrug or a prodrug, said linker being cleaved under physiological conditions to generate said therapeutically active form of A; and B represents a moiety which, when linked to A, results in the agent having a lower solubility than the therapeutically active form of A. In certain embodiments, the linkage L is hydrolyzed in bodily fluid. In certain embodiments, the linkage L is enzymatically cleaved. Examples of linkages which can be used include one or more hydrolyzable groups selected from the group consisting of an ester, an amide, a carbamate, a carbonate, a cyclic ketal, a thioester, a thioamide, a thiocarbamate, a thiocarbonate, a xanthate and a phosphate ester.

Other embodiments of the present invention provide a sustained release formulation comprising a polymer matrix and a therapeutically effective amount of an agent, dispersed in the polymer, having a general formula of A::B in which A represents a drug moiety having a therapeutically active form for producing a clinical response in a patient; :: represents a ionic bond between A and B that dissociates under physiological conditions to generate said therapeutically active form of A; and B represents a moiety which, when ionically bonded to A, results in the agent having a lower solubility than the therapeutically active form of A.

In certain embodiments, the solubility of therapeutically active form of A in water is greater than 1 mg/ml and the solubility of the agent in water is less than 1 mg/ml, and even more preferably less than 0.1 mg/ml, 0.01 mg/ml or even less than 0.001 mg/ml.

In other embodiments, the therapeutically active form of A is at least 10 times more soluble in water relative to the agent, and even more preferably at least 100, 1000 or even 10,000 times more soluble in water relative to the agent.

In certain preferred embodiments, when disposed in biological fluid (such as serum, synovial fluid, cerebral spinal fluid, lymph, urine, etc.), the sustained release formulation provides sustained release of the therapeutically active form of the agent for a period of at least 3 hours, and over that period of release, the concentration of the agent in fluid immediately outside the polymer is less than 10% of the concentration of the unreleased agent, and even more preferably less than 5%, 1% or even 0.1% of the concentration of the unreleased agent.

In certain embodiments, the duration of release from the polymer matrix of the agent is at least 3 hours, and even more preferably may be at least 24, 72, 100, 250, 500 or even 750 hours. In certain embodiments, the duration of release of the agent from the polymer matrix is at least one week, more preferably two weeks, or even more preferably at least three weeks. In certain embodiments, the duration of release of the agent from the polymer matrix is at least one month, more preferably two months, and even more preferably six months.

In certain embodiments, the therapeutically active form of A may have a LogP value at least 1 LogP unit less than the LogP value of the agent, and even more preferably at least 2, 3 or even 4 LogP unit less than the LogP value of the agent.

In certain embodiments, the prodrug, in its linked form, has an ED₅₀ for producing the clinical response at least 10 times greater than the ED₅₀ of the therapeutically active form of A, and even more preferably at least 100, 1000 or even 10,000 times greater than the ED₅₀ of the therapeutically active form of A. That is, in many embodiments, the agent per se is inert with respect to inducing the clinical response.

In certain embodiments, B is a hydrophobic aliphatic moiety.

In some instances, B is a drug moiety having a therapeutically active form generated upon cleavage of said linker L or dissociation of said ionic bond, and may be the same drug or a different drug than A.

In other embodiments, B, after cleavage from the prodrug, is a biologically inert moiety.

In certain embodiments, the pro-drug has an EC₅₀ at least 10 times greater than the EC₅₀ of the therapeutically active form of A. In preferred embodiments, the pro-drug has an EC₅₀ at least 100 times, or more preferably at least 1000 times, greater than the EC₅₀ of the therapeutically active form of A.

In some embodiments, the therapeutically active form of A is at least 10 times more soluble in water relative to said pro-drug. In preferred embodiments, the therapeutically active form of A is at least 100 times, or more preferably at least 1000 times, more soluble in water relative to said prodrug.

The A (and optionally B) moiety can be selected from amongst such drugs as immune response modifiers, anti-proliferatives, corticosteroids, angiostatic steroids, anti-parasitic drugs, anti-glaucoma drugs, antibiotics, anti-sense compounds, differentiation modulators, antiviral drugs, anticancer drugs, and non-steroidal anti-inflammatory drugs.

In certain embodiments, the polymer matrix is non-bioerodible, while in other embodiments it is bioerodible. Exemplary non-bioerodible polymer matrices can be formed from polyurethane, polysilicone, poly(ethylene-co-vinyl acetate) (EVA), polyvinyl alcohol, and derivatives and copolymers thereof.

Exemplary bioerodible polymer matrices can be formed from polyanhydride, polylactic acid, polyglycolic acid, polyorthoester, polyalkylcyanoacrylate, and derivatives and copolymers thereof.

In certain embodiments, the polymer matrix is chosen so as reduce interaction between the agent in the matrix and proteinaceous components in surrounding bathing fluid, e.g., by forming a matrix have physical (pore size, etc.) and/or chemical (ionized groups, hydrophobicity, etc.) characteristics which exclude proteins and peptides from the inner matrix, e.g., exclude proteins of greater than 100 kD, and even more preferably exclude proteins greater in size than 50 kD, 25 kD, 10 kD oreven 5 kD.

In preferred embodiments, diffusion through the polymer matrix is primarily non-release-rate-limiting with respect to the rate of release of the agent from the matrix. In certain embodiments, the polymer matrix is essentially non-release rate limiting with respect to the rate of release of the agent (e.g., the therapeutically active form of A) from the matrix.

In other embodiments, the subject polymer matrix influences the rate of release. For instance, the matrix can be derived to have charge or hydrophobicity characteristics which favor sequestration of the agent over released constituents (A and B). The polymer matrix can create a microenviromnent having a pH different than the bathing bodily fluid, such that hydrolysis and/or solubility of the agent is different within the matrix than in the surrounding fluids. In such a manner, the polymer can influence the rate of release, and the rate of hydrolysis of the agent, by differential electronic, hydrophobic or chemical interactions with the agent.

In certain embodiments, at least one of A or B is an antineoplastic agent. Exemplary antineoplastic agents include anthracyclines, vinca alkaloids, purine analogs, pyrimidine analogs, inhibitors of pyrimidine biosynthesis, and/or alkylating agents. Exemplary antineoplastic agents also include 5-fluorouracil (5FU), 5′-deoxy-5-fluorouridine 5-fluorouridine, 2′-deoxy-5-fluorouridine, fluorocytosine, 5-trifluoromethyl-2′-deoxyuridine, arabinoxyl cytosine, cyclocytidine, 5-aza-2′-deoxycytidine, arabinosyl 5-azacytosine, 6-azacytidine, N-phosphonoacetyl-L-aspartic acid, pyrazofurin, 6-azauridine, azaribine, 3-deazauridine, arabinosyl cytosine, cyclocytidine, 5-aza-2′-deoxycytidine, arabinosyl 5-azacytosine, 6-azacytidine, cladribine, 6-mercaptopurine, pentostatin, 6-thioguanine, and fludarabine phosphate.

In certain embodiments, the antineoplastic drug is a fluorinated pyrimidine, and even more preferably 5-FU, e.g., A is preferably 5-FU in certain embodiments.

In certain embodiments, at least one of A or B is an anti-inflammatory agent, such as, to illustrate, a non-steroidal anti-inflammatory (such as diclofenac, fenoprofen, flurbiprofen, ibuprofen, ketoprofen, ketorolac, naproxen, piroxicam and the like), a glucocorticoid (such as aclometasone, beclomethasone, betamethasone, budesonide, clobetasol, clobetasone, cortisone, desonide, desoximetasone, diflorosane, flumethasone, flunisolide, fluocinolone acetonide, fluocinolone, fluocortolone, fluprednidene, flurandrenolide, fluticasone, hydrocortisone, methylprednisolone aceponate, mometasone furdate, prednisolone, prednisone, rofleponide, and the like), or a steroidal anti-inflammatory (such as flucinolone acetonide (FA), or triamcinolone acetonide (TA)).

In some embodiments, A is an antineoplastic fluorinated pyrimidine, such as 5-fluorouracil, and B is an anti-inflammatory, such as fluocinolone acetonide, triamcinolone acetonide, diclofenac, or naproxen.

In some embodiments, the agent is selected from 5FU covalently bonded to FA (I), 5FU covalently bonded to naproxen (II), and 5FU covalently bonded to diclofenac (III). Exemplary agents include:

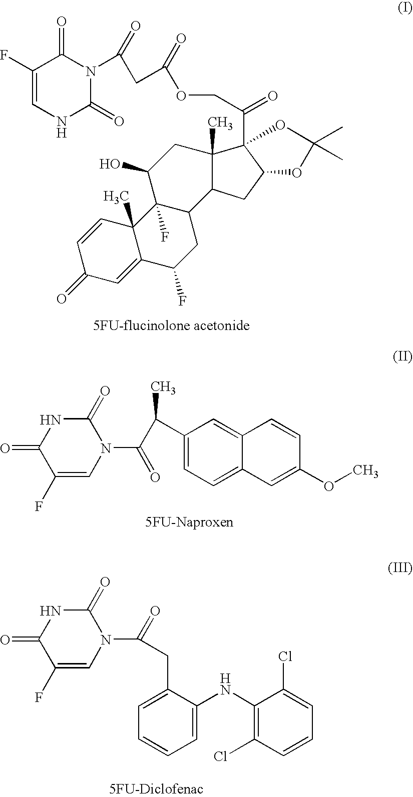

Another aspect of the invention relates to coated medical devices. For instance, in certain embodiments, the subject invention provides a medical device having a coating adhered to at least one surface, wherein the coating includes the subject polymer matrix and a therapeutically effective amount of an agent. Such coated devices can be implanted into a patient. In certain embodiments, the release rate of the agent can be controlled by varying the amount of agent dispersed in the matrix. Such coatings can also be applied to surgical implements such as screws, plates, washers, prosthesis, prosthesis anchors, tacks, staples, electrical leads, valves, membranes, radiation seeds. The devices can be catheters, implantable vascular access ports, blood storage bags, blood tubing, central venous catheters, arterial catheters, vascular grafts, intraaortic balloon pumps, heart valves, artificial hearts, a pacemaker, ventricular assist pumps, extracorporeal devices, blood filters, hemodialysis units, hemoperfasion units, plasmapheresis units, and filters adapted for deployment in a blood vessel.

In certain embodiments, the subject coatings are applied to a vascular stent. In certain instances, particularly where the stent is an expandable stent, the coating is flexible to accommodate compressed and expanded states of the stent.

In certain embodiments, the weight of the coating attributable to the agent is in the range of about 0.05 mg to about 50 mg of agent per cm² of the surface coated with said polymer matrix, and even more preferably 5 to 25 mg/cm².

In certain embodiments, the coating has a thickness that in the range of 5 micrometers to 100 micrometers.

In certain embodiments, the agent is present in the coating in an amount between 5% and 70% by weight of the coating, and even more preferably 25% to 50% by weight.

Yet another aspect of the invention provides a method for treating an intraluminal tissue of a patient. In general, the method comprising:

(a) providing a stent having an interior surface and an exterior surface, said stent having a coating on at least a part of the interior surface, the exterior surface, or both; said coating comprising a pharmaceutical agent in a biologically-tolerated polymer;

(b) positioning the stent at an appropriate intraluminal tissue site; and

(c) deploying the stent.

Another aspect of the invention relates to a coating composition for use in delivering a medicament from the surface of a medical device positioned in vivo. The composition comprises a polymer matrix and pharmaceutical agent as described above. The coating composition can be provided in liquid or suspension form for application to the surface of a medical device by spraying and/or dipping the device in the composition. In other embodiments, the coating composition is provided in powdered form and, upon addition of a solvent, can reconstitute a liquid or suspension form for application to the surface of a medical device by spraying and/or dipping the device in the composition.

Another aspect of the invention relates to an injectable composition for use in delivering a medicament to a patient. The composition includes a polymer matrix and agent as described above, and is provided in suspension form adapted for delivery by injection through a needle.

Additional advantages of the present invention will become readily apparent to those skilled in the art from the following detailed description, wherein only a preferred embodiment of the invention is shown and described by way of illustration of the best mode contemplated for carrying out the invention. As will be realized, the present invention is capable of other and different embodiments, and its several details are capable of modifications in various respects, all without departing from the scope of the present invention. Accordingly, the drawings and description are to be regarded as illustrative in nature, and not as restrictive.

### BRIEF DESCRIPTION OF THE DRAWINGS

FIG. 1 is a time-dependent graph of the release of a prodrug from a polymer-prodrug dispersion according to the present invention.

FIG. 2 is a time-dependent graph of the release of a prodrug from a polymer-prodrug dispersion according to the present invention.

FIG. 3 is a side plan view of a non-deployed stent according to the present invention.

FIG. 4 is a side plan view of a deployed stent according to the present invention.

### DETAILED DESCRIPTION OF THE INVENTION

I. Definitions

The term “active” as used herein means therapeutically or pharmacologically active.

The term “agent” as used herein is synonynymous with “compound” and means a drug, codrug, or prodrug thereof.

The term “ED₅₀” means the dose of a drug that produces 50% of its maximum response or effect.

The terms “granule”, “particle”, or “particulate” as used herein are used interchangeably and refer to any particle. In certain exemplary embodiments, the particles have a diameter in the range of about 0.01 mm to about 3 mm, preferably in the range of about 0.1 mm to about 2 mm, or even more preferrably in the range of about 0.3 mm to about 1.5 mm.

As used herein, the term “EC₅₀” means the concentration of a drug that produces 50% of its maximum response or effect. The term “IC₅₀” means the dose of a drug that inhibits a biological activity by 50%.

The term “LD₅₀” means the dose of a drug that is lethal in 50% of test subjects.

The term “therapeutic index” refers to the therapeutic index of a drug defined as LD₅₀/ED₅₀.

A “patient” or “subject” to be treated by the subject method can mean either a human or non-human animal.

“Physiological conditions” describe the conditions inside an organism, i.e., in vivo. Physiological conditions include the acidic and basic environments of body cavities and organs, enzymatic cleavage, metabolism, and other biological processes, and preferably refer to physiological conditions in a vertebrate, such as a mammal.

In general, “low solubility” means that the agent is only very slightly soluble in aqueous solutions having pH in the range of about 5 to about 8, and in particular to physiologic solutions, such as blood, blood plasma, etc. Some agents, e.g., low-solubility agents, will have solubilities of less than about 1 mg/ml, less than about 100 μg/ml, preferably less than about 20 μg/ml, more preferably less than about 15 μg/ml, and even more preferably less than about 10 μg/ml. Solubility is in water at a temperature of 25° C. as measured by the procedures set forth in the 1995 USP, unless otherwise stated. This includes compounds which are slightly soluble (about 10 mg/ml to about 1 mg/ml), very slightly soluble (about 1 mg/ml to about 0.1 mg/ml) and practically insoluble or insoluble compounds (less than about 0.1 mg/ml, preferably less than about 0.01 mg/ml).

As used herein, an agent's “LogP” refers to the logarithm of P (Partition Coefficient), where P is a measure of how well the agent partitions between octanol and water. P itself is a constant, defined as the ratio of concentration of compound in aqueous phase to the concentration of compound in octanol according to the following:

Partition Coefficient, P=[Organic]/[Aqueous] where [ ]=concentration

LogP=log₁₀(Partition Coefficient)=log₁₀P

A LogP value of 1 means that the concentration of the compound is ten times greater in the organic phase than in the aqueous phase. The increase in a LogP value of 1 indicates a ten-fold increase in the concentration of the compound in the organic phase as compared to the aqueous phase.

The term “codrug” as used herein means a compound, comprising a first molecule residue associated with a second molecule residue, wherein each residue, in its separate form (e.g., in the absence of the association), is biologically active, or a prodrug form of a biologically active compound. In preferred embodiments, either one or both of the first and second molecule residues are small molecules. The association between said residues can be either ionic or covalent and, in the case of covalent associations, either direct or indirect through a linker. The first molecule can be the same or different from the second. Exemplary formulae for co-drugs can be seen in formulae I, Ia, II, IIa, III, IIIa, and IV,

A₁*(—L—A₂*)ₙ (I)

A₁*(—A₂*)ₙ (Ia)

A₁*—L—A₂* (II)

A₁*—A₂* (IIa)

A₂*—L—A₁*—L—A₂* (III)

A₂*—A₁*—A₂* (IIIa)

wherein each of A₁*, A₂*, and L are defined as follows:

A₁* is a residue of a first biologically active compound, A₁;

A₂* is a residue of a second biologically active compound, A₂, which may be the same as or different from A₁;

L is a linking group selected from a direct bond and a divalent organic linking group; and

n is an integer having a value of from 1 to 4, preferably 1.

The term “prodrug” as used herein means a first molecule residue associated with a second molecule residue, wherein one of the residues is biologically active. In preferred embodiments, either one or both of the first and second molecule residues are small molecules. In some embodiments, one of the residues is not biologically active; in some embodiments the prodrug may be biologically inactive in its prodrug form. The association between said residues is covalent and can be either direct or indirect through a linker. Prodrugs of biologically active compounds include esters, as well as anhydrides, amides, and carbamates that are hydrolyzed in biological fluids to produce the parent compounds.

The term “physiological pH,” as used herein, refers to a pH that is about 7.4 at the standard physiological temperature of 37.4° C. The term “non-physiological pH,” as used herein, refers to a pH that is less than or greater than “physiological pH,” preferably between about 4 and 7.3, or greater than 7.5 and less than about 12. The term “neutral pH,” as used herein, refers to a pH of about 7. In preferred embodiments, physiological pH refers to pH 7.4, and non-physiological pH refers to pH between about 6 and 7. The term “acidic pH” refers to a pH that is below pH 7, preferably below about pH 6, or even below about pH 4.

The phrase “pharmaceutically acceptable carrier” as used herein means a pharmaceutically acceptable material, composition or vehicle, such as a liquid or solid filter, diluent, excipient, solvent or encapsulating material, involved in carrying or transporting a subject drug from one organ, or portion of the body, to another organ, or portion of the body. Each carrier must be “acceptable” in the sense of being compatible with other ingredients of the formulation and not injurious to the patient.

Some examples of materials which can serve as pharmaceutically acceptable carriers include (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

According to the present invention, the phrase “limited primarily by” when used to refer to the release rate of an agent, means that the rate of dissolution of the agent from the granule(s) into the matrix is lower than the rate of diffusion through the matrix or the rate of dispersion of the agent in the surrounding fluid, e.g., by at least a factor of three, preferably by at least a factor of five, ten, or even of 100. Thus, the rates of diffusion and dispersion are not the most influential factors in determining the rate of release of the agent from the formulation

II. Exemplary Embodiments

The present invention provides a drug delivery system that can provide various release profiles, e.g., varying doses and/or varying lengths of time. The present invention thereby addresses the need for an insertable, injectable, inhalable, implantable, or otherwise administrable drug delivery system that provides controlled time-release kinetics of drug, particularly in the vicinity of a desired locus of drug activity, while avoiding complications associated with prior art devices.

The present invention includes a sustained-release formulation comprising a therapeutically effective amount of at least one agent dispersed in a polymer matrix, wherein the agent is in granular or particulate form, e.g., as a plurality of granules. In certain embodiments, the sustained release occurs as the agent in the granules or particles dissolves into the polymer matrix, diffuses through the matrix, then is released into the surrounding physiological fluid. In certain embodiments, the sustained release may occur as the agent dissolves into the polymer matrix, diffuses through the matrix, and is released into physiological fluid that has absorbed into the polymer. According to the present invention, the steps of diffusion through the matrix and dispersion in the surrounding physiological fluid are primarily non-rate-limiting with respect to the rate of release of the agent from the formulation. The rate of release of the agent from the matrix is limited primarily by the rate at which the agent in the granules or particles dissolves into the matrix.

In some embodiments according to the present invention, the agent is a low-solubility pharmaceutical prodrug. Multiple agents may also be used.

It is preferred that the agent be relatively insoluble in the polymer matrix. In certain embodiments, the agent has a solubility in the polymer matrix of about 10 mg/ml or less, in other embodiments the agent solubility in the polymer matrix is about 1 mg/ml or less, or even about 0.1 mg/ml or less, or about 0.01 mg/ml or less. Preferably, the agent may possess a finite solubility with respect to the polymer matrix and still be within the scope of the present invention. In any event, an agent's solubility in the polymer matrix should be such that the agent will remain in substantially granular form within the polymer matrix.

The system of the present invention may include a polymer and a low solubility agent dispersed in the polymer. The polymer is permeable to the agent and is primarily not release-rate-limiting with respect to the rate of release of the agent from the polymer, and thus provides sustained release of the drug.

Once administered, the system gives a continuous supply of the agent to the desired locus of activity without necessarily requiring additional invasive penetrations into these regions. Instead, the system remains in the body and serves as a continuous source of the agent to the affected area. The system according to the present invention permits prolonged release of drugs over a specific period of days, weeks, months (e.g., about 3 months to about 6 months) or years (e.g., about 1 year to about 20 years, such as from about 5 years to about 10 years) until the agent is used up.

In certain embodiments, an intraluminal medical device may be used, with such device comprising the sustained release drug delivery coating. For example, such a coating may be applied to a stent via a conventional coating process, such as impregnating coating, spray coating and dip coating.

In one embodiment, an intraluminal medical device comprises an elongate radially expandable tubular stent having an interior luminal surface and an opposite exterior surface extending along a longitudinal stent axis. The stent may include a permanent implantable stent, an implantable grafted stent, or a temporary stent, wherein the temporary stent is defined as a stent that is expandable inside a vessel and is thereafter retractable from the vessel. The stent configuration may comprise a coil stent, a memory coil stent, a Nitinol stent, a mesh stent, a scaffold stent, a sleeve stent, a permeable stent, a stent having a temperature sensor, a porous stent, and the like. The stent may be deployed according to conventional methodology, such as by an inflatable balloon catheter, by a self-deployment mechanism (after release from a catheter), or by other appropriate means. The elongate radially expandable tubular stent may be a grafted stent, wherein the grafted stent is a composite device having a stent inside or outside of a graft. The graft may be a vascular graft, such as an ePTFE graft, a biological graft, or a woven graft.

In certain embodiments, the agent may be incorporated onto or affixed to the stent in a number of ways. For example, the agent may be directly incorporated into a polymeric matrix and sprayed onto the outer surface of a stent. The drug combination elutes from the polymeric matrix over time and enters the surrounding tissue. The drug combination preferably remains on the stent for at least three days up to approximately six months, and more preferably between seven and thirty days.

Upon dispersion in the immediately surrounding fluid, the agent is preferably immediately physiologically active. In certain embodiments, preferably those using codrugs or prodrugs, the agent may be slowly dissolved in physiologic fluids, but is relatively quickly dissociated into at least one pharmaceutically active compound upon dissolution in physiologic fluids. In some embodiments, the dissolution rate of the agent is in the range of about 0.001 μg/day to about 20 μg/day, preferably to about 10 μg/day. In certain embodiments, the agent has dissolution rates in the range of about 0.01 to about 1 μg/day. In particular embodiments, the agent has a dissolution rate of about 0.1 μg/day.

The pharmaceutical agent is incorporated into a biocompatible (i.e., biologically tolerated) polymer vehicle. In some embodiments according to the present invention, the agent is present as a plurality of granules dispersed within the polymer vehicle. In such cases, it is preferred that the agent be relatively insoluble in the polymer vehicle, however the agent may possess a finite solubility with respect to the polymer vehicle and still be within the scope of the present invention. In either case, the polymer vehicle solubility of the agent should be such that the agent will disperse throughout the polymer vehicle, while remaining in substantially granular form. The polymer according to the present invention comprises any biologically tolerated polymer that is permeable to the agent, so long as the permeability is not the principal rate-determining factor in the rate of release of the agent from the polymer.

In preferred embodiments, the polymer is a hydrogel, such as the hydrogels described by Hennink et al in U.S. Pat. No. 6,497,903, the teachings of which are incorporated by reference herein. The hydrogel may contain one or more functional groups having the ability to form linkers to other polymers, e.g., dextran or derivatized dextrans. The hydrogel may be applied in multiple layers; it may also have acidic or basic functional groups by which the pH of the matrix microenvironment may be controlled. For example, the addition of an acidic functional group would increase the acidity of the matrix. By controlling the pH of the matrix, the pH of the agent may also be controlled, thereby stabilizing the agent. In certain embodiments, controlling the pH of the matrix maintains the agent in a non-ionized form within the polymer matrix.

In cases where the agent is a codrug or prodrug, controlling the matrix pH enables the modulation of codrug or prodrug cleavage, such that cleavage may selectively occur either before or after the agent is released from the matrix. Near zero-order kinetics may be achieved (such that the rate of release is nearly approximately linear with respect to time) in cases where cleavage occurs after a codrug or prodrug is released from the matrix.

In some embodiments according to the present invention, the polymer is non-bioerodible. As noted above, exemplary non-bioerodible polymers include polysilicone, EVA, polyvinyl alcohol, polyurethane (such as polycarbonate-based polyurethane), and derivatives and copolymers thereof.

In other embodiments of the present invention, the polymer is bioerodible. As previously noted, examples of bioerodible polymers useful in the present invention include polyanhydride, polylactic acid, polyglycolic acid, polyorthoester, polyalkylcyanoacrylate or derivatives and copolymers thereof.

Other suitable polymers include naturally occurring (e.g., those derived from collagen, hyaluronic acid, etc.) or synthetic materials that are biologically compatible with bodily fluids and mammalian tissues, and essentially insoluble in bodily fluids with which the polymer will come in contact. Certain exemplary polymers include polysilicone.

Other suitable polymers include polypropylene, polyester, polyethylene vinyl acetate, polyethylene oxide (PEO), polypropylene oxide, polycarboxylic acids, polyalkylacrylates, cellulose ethers, silicone, poly(dl-lactide-co glycolide), various Eudragrits (for example, NE30D, RS PO and RL PO), polyalkyl-alkyacrylate copolymers, polyester-polyurethane block copolymers, polyether-polyurethane block copolymers, polydioxanone, poly-(β-hydroxybutyrate), polylactic acid (PLA), polycaprolactone, polyglycolic acid, and PEO-PLA copolymers.

In some embodiments according to the invention, the system comprises a polymer that is relatively rigid. In other embodiments, the system comprises a polymer that is soft and malleable. In still other embodiments, the system includes a polymer that has an adhesive character. In other embodiments, the system includes a polymer that is a hydrogel, or a polymer that is a biocompatible fluid or a semi-solid (such as long-chain polyethylene glycol). Hardness, elasticity, adhesive, and other characteristics of the polymer are widely variable, depending upon the particular final physical form of the system, as discussed in more detail below.

The skilled artisan will understand that the polymer according to the present invention is prepared under conditions suitable to impart permeability such that it is not the principal rate-determining factor in the release of the agent from the polymer. In addition, the suitable polymers essentially prevent interaction between the agent dispersed/suspended in the polymer and proteinaceous components in the bodily fluid. The use of rapidly dissolving polymers or polymers highly soluble in bodily fluid or which permit interaction between the agent and proteinaceous components are to be avoided in certain instances, since dissolution of the polymer or interaction with proteinaceous components could affect the rate of drug release.

The coating of the invention may be formed by mixing one or more suitable monomers and a suitable pharmaceutical agent, then polymerizing the monomer to form the polymer system. In this way, the agent may be dispersed in the polymer. In other embodiments, the agent is mixed into a liquid polymer or polymer dispersion and then the polymer/agent suspension is further processed to form the inventive coating. Suitable further processing may include crosslinking with suitable crosslinking agents, further polymerization of the liquid polymer or polymer dispersion, copolymerization with a suitable monomer, block copolymerization with suitable polymer blocks, etc. The further processing traps the drug in the polymer so that the agent is suspended or dispersed in the polymer vehicle.

Any number of non-erodible polymers may be utilized in conjunction with the invention. Film-forming polymers that can be used for coatings in this application can be absorbable or non-absorbable and must be biocompatible to minimize irritation. The polymer may be either biostable or bioabsorbable depending on the desired rate of release or the desired degree of polymer stability, but a bioabsorbable polymer may be preferred since, unlike biostable polymer, it will not be present long after implantation or other administration to cause any adverse, chronic local response. Furthermore, bioabsorbable polymers do not present the risk that over extended periods of time there could be an adhesion loss between a delivery device and coating caused by the stresses of the biological environment that could dislodge the coating and introduce further problems even after the device is encapsulated in tissue.

Suitable film-forming bioabsorbable polymers that could be used include polymers selected from the group consisting of aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, biomolecules and blends thereof. For the purpose of this invention aliphatic polyesters include homopolymers and copolymers of lactide (which includes lactic acid d-,1- and meso lactide), ε-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6, 6-dimethyl-1,4-dioxan-2-one and polymer blends thereof. Poly(iminocarbonate) for the purpose of this invention include as described by Kemnitzer and Kohn, in the Handbook of Biodegradable Polymers, edited by Domb, Kost and Wisemen, Hardwood Academic Press, 1997, pages 251-272. Copoly(ether-esters) for the purpose of this invention include those copolyester-ethers described in Journal of Biomaterials Research, Vol. 22, pages 993-1009, 1988 by Cohn and Younes and Cohn, Polymer Preprints (ACS Division of Polymer Chemistry) Vol. 30(1), page 498, 1989 (e.g., PEO/PLA). Polyalkylene oxalates for the purpose of this invention include U.S. Pat. Nos. 4,208,511; 4,141,087; 4,130,639; 4,140,678; 4,105,034; and 4,205,399 (incorporated by reference herein). Polyphosphazenes, co-, ter- and higher order mixed monomer based polymers made from L-lactide, D,L-lactide, lactic acid, glycolide, glycolic acid, para-dioxanone, trimethylene carbonate and ε-caprolactone such as are described by Allcock in The Encyclopedia of Polymer Science, Vol. 13, pages 31-41, Wiley Intersciences, John Wiley & Sons, 1988 and by Vandorpe, Schacht, Dejardin and Lemmouchi in the Handbook of Biodegradable Polymers, edited by Domb, Kost and Wisemen, Hardwood Academic Press, 1997, pages 161-182 (which are hereby incorporated by reference herein). Polyanhydrides from diacids of the form HOOC—C₆H₄—O—(CH₂)ₘ—O—C₆H₄—COOH where m is an integer in the range of from 2 to 8 and copolymers thereof with aliphatic alpha-omega diacids of up to 12 carbons. Polyoxaesters polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213 and 5,700,583; (which are incorporated herein by reference). Polyorthoesters such as those described by Heller in Handbook of Biodegradable Polymers, edited by Domb, Kost and Wisemen, Hardwood Academic Press, 1997, pages 99-118 (hereby incorporated herein by reference). Film-forming polymeric biomolecules for the purpose of this invention include naturally occurring materials that may be enzymatically degraded in the human body or are hydrolytically unstable in the human body such as fibrin, fibrinogen, collagen, elastin, and absorbable biocompatable polysaccharides such as chitosan, starch, fatty acids (and esters thereof), glucoso-glycans and hyaluronic acid.

Suitable film-forming biostable polymers with relatively low chronic tissue response, such as polyurethanes, silicones, poly(meth)acrylates, polyesters, polyalkyl oxides (polyethylene oxide), polyvinyl alcohols, polyethylene glycols and polyvinyl pyrrolidone, as well as, hydrogels such as those formed from crosslinked polyvinyl pyrrolidinone and polyesters could also be used. Other polymers could also be used if they can be dissolved, cured or polymerized on a stent or other relevant delivery device. These include polyolefins, polyisobutylene and ethylene-alphaolefin copolymers; acrylic polymers (including methacrylate) and copolymers, vinyl halide polymers and copolymers, such as polyvinyl chloride; polyvinyl ethers, such as polyvinyl methyl ether; polyvinylidene halides such as polyvinylidene fluoride and polyvinylidene chloride; polyacrylonitrile, polyvinyl ketones; polyvinyl aromatics such as polystyrene; polyvinyl esters such as polyvinyl acetate; copolymers of vinyl monomers with each other and olefins, such as etheylene-methyl methacrylate copolymers, acrylonitrile-styrene copolymers, ABS resins and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate, cellulose, cellulose acetate, cellulose acetate butyrate; cellophane; cellulose nitrate; cellulose propionate; cellulose ethers (e.g., carboxymethyl cellulose and hydroxyalkyl celluloses); and combinations thereof. Polyamides for the purpose of this application would also include polyamides of the form —NH—(CH₂)ₙ—CO—and NH—(CH₂)ₓ—NH—CO—(CH₂)_{y}—CO, wherein n is preferably an integer in the range of from 6 to 13; x is an integer in the range of from 6 to 12; and y is an integer in the range of from 4 to 16. The list provided above is illustrative but not limiting.

If used as a coating for a device (e.g., a stent), the polymers also should adhere to the device and should not be so readily deformable after deposition on the device as to be able to be displaced by hemodynamic stresses. The polymer's molecular weight should be high enough to provide sufficient toughness so that the polymers will not be rubbed off during handling or deployment of the device and will not crack during expansion (thermal or physical) of the device. In certain embodiments, the polymer has a melting temperature above 40° C., preferably above about 45° C., more preferably above 50° C. and most preferably above 55° C.

Coating may be formulated by mixing one or more agent with one or more coating polymers in a coating mixture. The therapeutic agent may be present as a liquid, a finely divided solid, or any other appropriate physical form. Optionally, the mixture may include one or more additives, e.g., nontoxic auxiliary substances such as diluents, carriers, excipients, stabilizers or the like. Other suitable additives may be formulated with the polymer and pharmaceutically active agent. For example, more hydrophilic polymers selected from the previously described lists of biocompatible film forming polymers may be added to a biocompatible hydrophobic coating to modify the release profile (or a more hydrophobic polymer may be added to a hydrophilic coating to modify the release profile). As an example, a hydrophilic polymer may be added to an aliphatic polyester coating to modify the release profile, wherein the hydrophilic polymer is selected from polyethylene oxide, polyvinyl pyrrolidone, polyethylene glycol, carboxylmethyl cellulose, hydroxymethyl cellulose, and combinations thereof. Appropriate relative amounts can be determined by monitoring the in vitro and/or in vivo release profiles for the therapeutic agents.

Essentially, the agent(s) elute from the matrix by dissolution from the granules into the matrix, diffusion through the polymer matrix, and dispersion into the immediately surrounding fluid. Polymers are permeable, thereby allowing solids, liquids and gases to escape therefrom. The total thickness of the polymeric matrix is in the range from about one micron to about twenty microns or greater. It is important to note that primer layers and metal surface treatments may be utilized before the polymeric matrix is affixed to or coated onto a medical device. For example, acid cleaning, alkaline (base) cleaning, salinization and parylene deposition may be used as part of the overall process described.

In certain embodiments, multiple coatings can be used. For instance, the various coatings can differ in the concentration of the agent, the identity of agent (active ingredients, linkers, etc.), the characteristics of the polymer matrix (composition, porosity, etc.) and/or the presence of other drugs or release modifiers.

To exemplify a process for preparing a device, a poly(ethylene-co-vinylacetate), polybutylmethacrylate and drug combination suspension may be incorporated into or onto a stent device in a number of ways. For example, the suspension may be sprayed onto a stent or the stent may be dipped into the suspension. Other methods include spin coating and RF plasma polymerization. In one exemplary embodiment, the suspension is sprayed onto the stent and then allowed to dry. In another exemplary embodiment, the suspension may be electrically charged to one polarity and the stent electrically charged to the opposite polarity. In this manner, the suspension and stent will be attracted to one another. In using this type of spraying process, waste may be reduced and more precise control over the thickness of the coat may be achieved.

In another exemplary embodiment, the agent may be incorporated into a film-forming polyfluoro copolymer comprising an amount of a first moiety selected from the group consisting of polymerized vinylidenefluoride and polymerized tetrafluoroethylene, and an amount of a second moiety other than the first moiety and which is copolymerized with the first moiety, thereby producing the polyfluoro copolymer, the second moiety being capable of providing toughness or elastomeric properties to the polyfluoro copolymer, wherein the relative amounts of the first moiety and the second moiety are effective to provide the coating and film produced therefrom with properties effective for use in treating implantable medical devices.

In one embodiment according to the present invention, the exterior surface of the expandable tubular stent of an intraluminal medical device comprises a coating according to the present invention. The exterior surface of a stent having a coating is the tissue-contacting surface and is biocompatible. The “sustained release drug delivery system coated surface” is synonymous with “coated surface”, which surface is coated, covered or impregnated with sustained release drug delivery system according to the present invention.

In an alternate embodiment, the interior luminal surface or entire surface (i.e. both interior and exterior surfaces) of an elongate radially expandable tubular stent of an intraluminal medical device has the coated surface. The interior luminal surface having the inventive sustained release drug delivery system coating is also the fluid contacting surface, and is biocompatible and blood compatible.

The process for making a surface coated stent includes deposition onto the stent of a coating by, for example, dip coating or spray coating. In the case of coating one side of the stent, only the surface to be coated is exposed to the dip or spray. The treated surface may be all or part of an interior luminal surface, an exterior surface, or both interior and exterior surfaces of the intraluminal medical device. The stent may be made of a porous material to enhance deposition or coating into a plurality of micropores on or in the applicable stent surface, wherein the microporous size is preferably about 100 microns or less.

U.S. Pat. No. 5,773,019, U.S. Pat. No. 6,001,386, and U.S. Pat. No. 6,051,576 disclose controlled-release devices and drugs and are incorporated in their entireties herein by reference.

Problems associated with treating restinosis and neointimal hyperplasia can be addressed by the choice of pharmaceutical agent used to coat the medical device. In certain embodiments of the present invention, the chosen pharmaceutical agent comprises at least two pharmaceutically active compounds. The pharmaceutically active compounds can be the same or different chemical species, and can be formed, as desired, in equi-molar or non-equi-molar concentrations to provide optimal treatment based on the relative activities and other pharmaco-kinetic properties of the compounds.

The drug combination, particularly where co-drug formulations are used, may itself be advantageously very slightly soluble, or even insoluble in physiologic fluids, such as blood and blood plasma, and has the property of regenerating any or all of the pharmaceutically active compounds when dissolved in physiologic fluids. In other words, to the extent that an agent dissolves in physiologic fluids, it is quickly and efficiently converted into the constituent pharmaceutically active compounds upon dissolution. However, while the low solubility of the pharmaceutical agent helps maintain the agent in the vicinity of an intraluminal lesion, the release rate of the agent from the matrix is not controlled by the dissolution of the agent in the surrounding fluid but, rather, by the rate of dissolution of the agent from the particles or granules into the matrix. In any event, the quick conversion of the pharmaceutical agent into the constituent pharmaceutically active compound or compounds insures a steady, controlled dose of the pharmaceutically active compounds near the site of the lesion to be treated.

As noted above, examples of suitable pharmaceutically active agents include immune response modifiers such as cyclosporin A and FK 506, corticosteroids such as dexamethasone, FA and TA, angiostatic steroids such as trihydroxy steroids, antibiotics including ciprofloxacin, differentiation modulators such as retinoids (e.g., trans-retinoic acid, cis-retinoic acid and analogues), anticancer/anti-proliferative prodrugs such as 5-FU and carmustine (BCNU), and non-steroidal anti-inflammatory prodrugs such as naproxen, diclofenac, indomethacin and flurbiprofen.

In some embodiments according to the present invention, the preferred first pharmaceutically active compound is 5-FU.

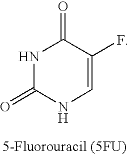

In some embodiments according to the present invention, the second pharmaceutically active compound is selected from FA, TA, diclofenac, and naproxen.

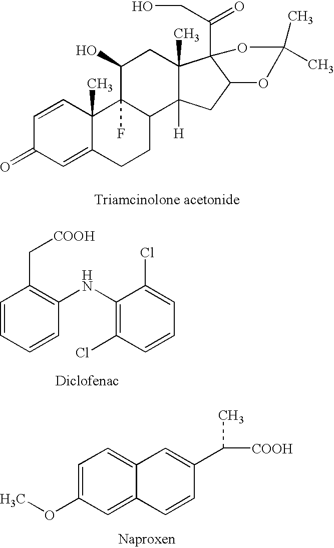

The pharmaceutically active agent may comprise further residues of pharmaceutically active compounds. Such further pharmaceutically active compounds include immune response modifiers such as cyclosporin A and FK 506, corticosteroids such as dexamethasone, FA and TA, angiostatic steroids such as trihydroxy steroids, antibiotics including ciprofloxacin, differentiation modulators such as retinoids (e.g., trans-retinoic acid, cis-retinoic acid and analogues), anticancer/anti-proliferative prodrugs such as 5-FU and BCNU, and non-steroidal anti-inflammatory prodrugs such as naproxen, diclofenac, indomethacin and flurbiprofen.

In certain embodiments, the agent comprises a moiety of at least two pharmaceutically active compounds that can be covalently bonded, connected through a linker, ionically combined, or combined as a mixture.

In some embodiments according to the present invention, first and second pharmaceutically active compounds are covalently bonded directly to one another. Where the first and second pharmaceutically active compounds are directly bonded to one another by a covalent bond, the bond may be formed by forming a suitable covalent linkage through an active group on each active compound. For instance, an acid group on the first pharmaceutically active compound may be condensed with an amine, an acid or an alcohol on the second pharmaceutically active compound to form the corresponding amide, anhydride or ester, respectively.

In addition to carboxylic acid groups, amine groups, and hydroxyl groups, other suitable active groups for forming linkages between pharmaceutically active moieties include sulfonyl groups, sulfhydryl groups, and the acid halide and acid anhydride derivatives of carboxylic acids.

In other embodiments, the pharmaceutically active compounds may be covalently linked to one another through an intermediate linker. The linker advantageously possesses two active groups, one of which is complementary to an active group on the first pharmaceutically active compound, and the other of which is complementary to an active group on the second pharmaceutically active compound. By ‘complementary’, it is meant that the groups can readily be linked through a covalent bond. For example, where the first and second pharmaceutically active compounds both possess free hydroxyl groups, the linker may suitably be a diacid, which will react with both compounds to form a diether linkage between the two residues. In addition to carboxylic acid groups, amine groups, and hydroxyl groups, other suitable active groups for forming linkages between pharmaceutically active moieties include sulfonyl groups, sulfhydryl groups, and the haloic acid and acid anhydride derivatives of carboxylic acids.

Suitable linkers are set forth in Table 1 below.

TABLE 1

| First Pharmaceutically | Second Pharmaceutically | |
|---|---|---|
| Active Compound Active | Active Compound Active | |
| Group | Group | Suitable Linker |
| Amine | Amine | Diacid |
| Amine | Hydroxy | Diacid |
| Hydroxy | Amine | Diacid |
| Hydroxy | Hydroxy | Diacid |
| Acid | Acid | Diamine |
| Acid | Hydroxy | Amino acid, |
| | | hydroxyalkyl |
| | | acid, |
| | | sulfhydrylalkyl |
| | | acid |
| Acid | Amine | Amino acid, |
| | | hydroxyalkyl |
| | | acid, |
| | | sulfhydrylalkyl |
| | | acid |

Suitable diacid linkers include oxalic, malonic, succinic, glutaric, adipic, pimelic, suberic, azelaic, sebacic, maleic, fumaric, tartaric, phthalic, isophthalic, and terephthalic acids. While diacids are named, the skilled artisan will recognize that in certain circumstances the corresponding acid halides or acid anhydrides (either unilateral or bilateral) are preferred as linker reprodrugs. A preferred anhydride is succinic anhydride. Another preferred anhydride is maleic anhydride. Other anhydrides and/or acid halides may be employed by the skilled artisan to good effect.

Suitable amino acids include y-butyric acid, 2-aminoacetic acid, 3-aminopropanoic acid, 4-aminobutanoic acid, 5-aminopentanoic acid, 6-aminohexanoic acid, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Again, the acid group of the suitable amino acids may be converted to the anhydride or acid halide form or otherwise activated to nucleophilic attack prior to their use as linker groups.

Suitable diamines include 1,2-diaminoethane, 1,3-diaminopropane, 1,4-diaminobutane, 1,5-diaminopentane, 1,6-diaminohexane.

Suitable aminoalcohols include 2-hydroxy-1-aminoethane, 3-hydroxy-1-aminoethane, 4-hydroxy-1-aminobutane, 5-hydroxy-1-aminopentane, 6-hydroxy-1-aminohexane.

Suitable hydroxyalkyl acids include 2-hydroxyacetic acid, 3-hydroxypropanoic acid, 4-hydroxybutanoic acid, 5-hydroxypentanoic acid, 5-hydroxyhexanoic acid.

The person having skill in the art will recognize that by selecting first and second pharmaceutical moieties (and optionally third, etc. pharmaceutical moieties) having suitable active groups, and by matching them to suitable linkers, a broad palette of inventive compounds may be prepared within the scope of the present invention.

As noted previously, xemplary pharmaceutically active agents include 5-FU covalently bonded to FA, 5-FU covalently bonded to diclofenac, 5-FU covalently bonded to TA, and 5-FU covalently bonded to naproxen.

Other exemplary codrugs include the following:

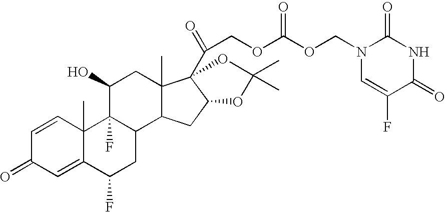

5-TC-70.1 (codrug of fluocinolone acetonide with 5-FU via formaldehyde linkage)

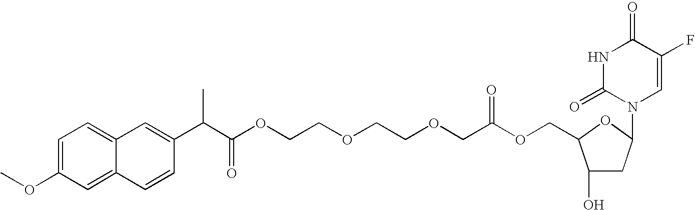

5-TC-63.1 (codrug of naproxen with floxuridine via oxa acid linkage)

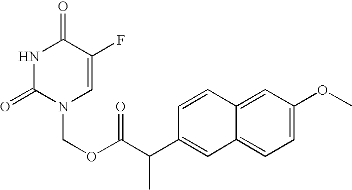

3-TC-112 (codrug of naproxen with 5-FU via formaldehyde linkage)

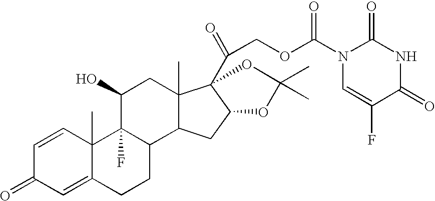

G-427.1 (direct codrug of triamcinolone acetonide with 5-FU)

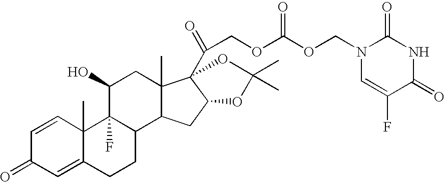

TC-32 (codrug of triamcinolone acetonide with 5-FU via formaldehyde linkage)

Some exemplary co-drugs which join the first and second pharmaceutically active compounds with different linkages include:

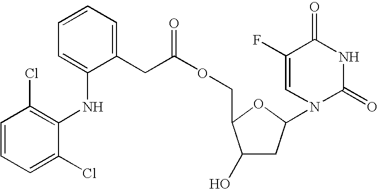

Co-drug of Floxuridine with Diclofenac (1:1)

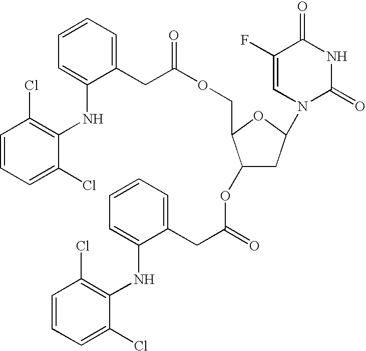

Co-drug of Floxuridine with Diclofenac (1:2)

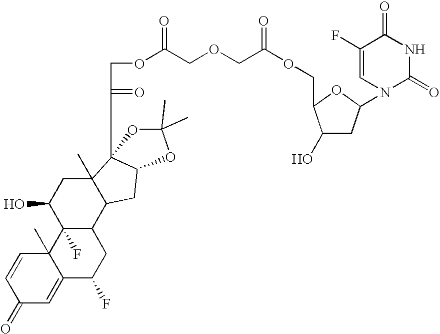

Co-drug of Floxuridine with Fluocinolone acetonide (1:1)

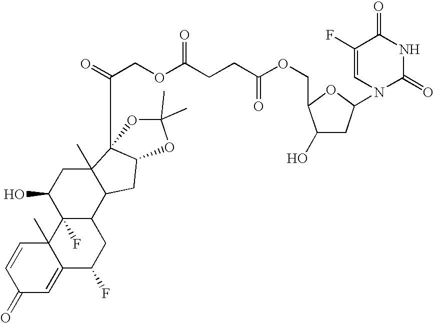

Co-drug of Floxuridine with Fluocinolone acetonide (1:1)

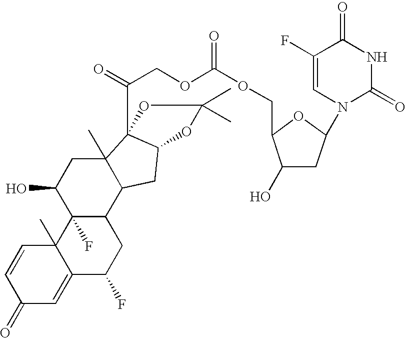

Co-drug of Floxuridine with Fluocinolone acetonide (1:1)

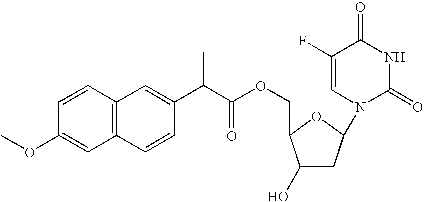

Co-drug of Floxuridine with Naproxen (1:1)

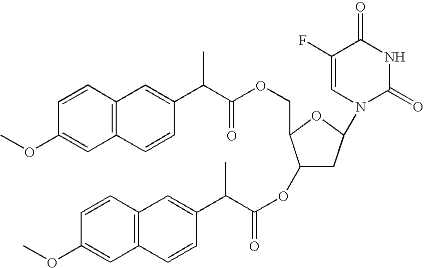

Co-drug of Floxuridine with Naproxen (1:2)

In other embodiments, the first and second pharmaceutically active compounds may be combined to form a salt. For instance, the first pharmaceutically active compound may be an acid, and the second pharmaceutically active compound may be a base, such as an amine. As a specific example, the first pharmaceutically active compound may be diclofenac or naproxen (acids), and the second pharmaceutically active compound may be ciprofloxacin (a base). The combination of diclofenac and ciprofloxacin would for instance form the salt:

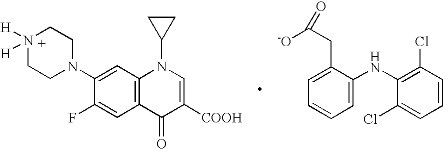

Ciprofloxacin-Diclofenac

The system of the present invention may be formed by mixing one or more suitable monomers and a suitable pharmaceutical agent, then polymerizing the monomer to form the polymer system. In this way, the agent is dissolved or dispersed in the polymer. In other embodiments, the agent is mixed into a liquid polymer or polymer dispersion and then the polymer is further processed to form the inventive system. Suitable further processing includes crosslinking with suitable crosslinking agents, further polymerization of the liquid polymer or polymer dispersion, copolymerization with a suitable monomer, block copolymerization with suitable polymer blocks, etc. The further processing traps the agent in the polymer so that the agent is suspended or dispersed in the polymer vehicle.

In some embodiments according to the present invention, monomers for forming a polymer are combined with an inventive compound and are mixed to make a dispersion of the inventive compound in the monomer suspension. The dispersion then may be applied to a stent or other device according to a conventional coating process, after which the crosslinking process is initiated by a conventional initiator, such as UV light. In other embodiments according to the present invention, a polymer composition is combined with an inventive compound to form a dispersion. The dispersion then may be applied to a stent or other device and the polymer is cross-linked to form a solid coating. In other embodiments according to the present invention, a polymer and a compound are combined with a suitable solvent to form a dispersion, which is then applied to a stent or other device in a conventional fashion. The solvent is then removed by a conventional process, such as heat evaporation, with the result that the polymer and inventive drug (together forming a sustained-release drug delivery system) remain on the device as a coating.

Embodiments of the system according to the present invention take many different forms. In some embodiments, the system consists of a pharmaceutical agent suspended or dispersed in the polymer. In certain other embodiments, the system consists of an agent and a semi-solid or gel polymer, which is adapted to be injected via a syringe into a body. In certain embodiments the system consists of an agent and a polymer that can be administered orally. In other embodiments according to the present invention, the system consists of a pharmaceutical agent and a soft, flexible polymer, which is adapted to be inserted or implanted into a body by a suitable surgical method. In still further embodiments according to the present invention, the system consists of a hard, solid polymer, which is adapted to be inserted or implanted into a body by a suitable surgical method. In additional embodiments of the present invention, the system comprises a polymer having a pharmaceutical agent suspended or dispersed therein which is suitable for inhalation. In further embodiments, the system comprises a polymer having the agent suspended or dispersed therein, wherein the agent and polymer mixture forms a coating on a surgical implement, such as a screw, stent, pacemaker, etc. In particular embodiments according to the present invention, the device consists of a hard, solid polymer, which is shaped in the form of a surgical implement such as a surgical screw, plate, stent, etc., or some part thereof. In still other embodiments, the system comprises a polymer that is a hydrogel as described above.

In some embodiments according to the present invention, provided is a medical device comprising a substrate having a surface, such as an exterior surface, and a coating on the exterior surface. The coating comprises a polymer and a pharmaceutical agent dispersed in the polymer, wherein the polymer is permeable to the agent and is primarily not release-rate-limiting with respect to the rate of release of the agent from the polymer. In certain embodiments according to the present invention, the device comprises an agent suspended or dispersed in a suitable polymer, wherein the agent and polymer are coated onto an entire substrate, e.g., a surgical implement. Such coating may be accomplished by spray coating or dip coating.

In other embodiments according to the present invention, the device comprises an agent and polymer suspension or dispersion, wherein the polymer is rigid, and forms a constituent part of a device to be inserted or implanted into a body. For instance, in particular embodiments according to the present invention, the device is a surgical screw, stent, pacemaker, etc., coated with the agent suspended or dispersed in the polymer. In other particular embodiments according to the present invention, the polymer in which the agent is suspended forms a tip or a head, or part thereof, of a surgical screw. In other embodiments according to the present invention, the polymer in which the agent is suspended or dispersed is coated onto a surgical implement such as surgical tubing (such as colostomy, peritoneal lavage, catheter, and intravenous tubing). In still further embodiments according to the present invention, the device is an intravenous needle having the polymer and agent (for instance, an agent of an anticoagulant such as heparin or codrug thereof) coated thereon.

In certain embodiments, a device containing a sustained release formulation comprising a plurality of granules is surrounded by ambient physiological tissue when applied to a physiological system (e.g. the device is inserted into a human body). In certain of such embodiments, at least a portion of the granules are directly exposed to the surrounding tissue.

As discussed above, a device according to the present invention comprises a polymer that is bioerodible or non-bioerodible. The choice of bioerodible versus non-bioerodible polymer is made based upon the intended end use of the system or device. In some embodiments according to the present invention, the polymer is advantageously bioerodible. For instance, the polymer is advantageously bioerodible for use in connection with a bioerodible device. In certain embodiments, the polymer is advantageously bioerodible for use in a coating on a surgically implantable device, such as a screw, stent, pacemaker, etc. Other embodiments according to the present invention in which the polymer is advantageously bioerodible include devices that are implantable, inhalable, or injectable suspensions or dispersions of one or more agents in a polymer, wherein the further elements (such as screws or anchors) are not utilized.

In some embodiments according to the present invention wherein the polymer is poorly permeable and bioerodible, the rate of bioerosion of the polymer is advantageously sufficiently slower than the rate of drug release so that the polymer remains in place for a substantial period of time after the drug has been released, but is eventually bioeroded and resorbed into the surrounding tissue. In other embodiments according to the present invention, the rate of bioerosion of the polymer occurs over a similar time frame as the drug release. In certain embodiments the rate of polymer bioerosion is advantageously on the same order as the rate of drug release. For instance, where the system comprises an agent suspended or dispersed in a polymer that is coated onto a surgical implement, such as an orthopedic screw, a stent, a pacemaker, the polymer advantageously bioerodes at such a rate that the surface area of the agent that is directly exposed to the surrounding body tissue remains substantially constant over time.

In some embodiments according to the present invention, the polymer is non-bioerodible, or is bioerodible only at a rate slower than a dissolution rate of the pharmaceutical agent, and the diameter of the agent's granules is such that when the coating is applied to a medical device, (e.g., a stent), the granules' surfaces are at least partially exposed to the ambient tissue. In such embodiments, the release rate of the pharmaceutical agent is proportional to the exposed surface area of the granules.

In other embodiments according to the present invention, the polymer vehicle is permeable to water in the surrounding tissue, e.g., in blood plasma. In such cases, water solution may permeate the polymer, thereby contacting the pharmaceutical agent. In preferred embodiments, the polymer matrix limits the interaction of the agent with elements (e.g., enzymes) present in the physiological media (such as stomach contents, blood plasma, and the like). For example, and without limitation, the matrix may be a diffusional barrier to the movement of peptides and/or proteins from the media into the matrix containing the agent.

In some embodiments according to the present invention, the polymer is non-bioerodible. Non-bioerodible polymers are especially useful where the system includes a polymer intended to be coated onto, or form a constituent part, of a surgical implement that is adapted to be permanently, or semi-permanently, inserted or implanted into a body. Exemplary devices in which the polymer advantageously forms a permanent coating on a surgical implement include an orthopedic screw, a stent, a prosthetic joint, an artificial valve, a pacemaker, etc.

A system according to the present invention (e.g., a surgical system) is used in a manner suitable for the desired therapeutic effect. For instance in some preferred embodiments according to the invention, the mode of administration is by injection. In such cases, the system is a liquid or gel, and is introduced into the desired locus by taking the system up into the barrel of a syringe and injecting the system through a needle into the desired locus. Such a mode of administration is suitable for intramuscular injection, for instance intramuscular injection of sustained-release formulations of microbicides, including antibiotics, antivirals, and steroids. This mode of administration is also useful where the desired therapeutic effect is the sustained release of hormones such as thyroid medication, birth control prodrugs, estrogen for estrogen therapy, etc. The skilled clinician will appreciate that this mode of administration is adaptable to various therapeutic areas, and will adapt the particular polymer and drug of the system to the desired therapeutic effect.

In embodiments according to the present invention in which the mode of administration is to be by injection, the system is advantageously a drug suspended or dispersed in a viscous polymer vehicle. The system is, in such cases, a stable suspension or dispersion of drug in liquid polymer vehicle. Advantageously, the polymer vehicle will be either non-bioerodible or will bioerode at a rate slower than the rate of dispension of the drug from the granules and into the matrix. In such cases, the system stays in place in place relative to the surrounding tissue, preventing the drug from being prematurely released into the surrounding tissue.

The precise properties of the system according to the present invention depend upon the therapeutic use intended, the physical state of the drug to be incorporated into the system under physiologic conditions, etc.

In some embodiments according to the present invention, the system is advantageously a solid device of a shape and form suitable for implantation, for instance subcutaneously, etc. In some embodiments according to the present invention, the system is in the shape of an elongated ovoid, and the polymer is a solid polymer whose permeability is such that it is not the primary rate-determining factor in the rate of release of the agent. In particular embodiments according to the present invention, the polymer is bioerodible. In other embodiments according to the present invention, the polymer is non-bioerodible.

In embodiments where the device comprises a substrate and a coating on the substrate, such as a screw, stent, pacemaker, prosthetic joint, etc., the device is used in substantially the manner of the corresponding prior art surgical implement. For instance, a device that comprises a screw coated with a composition comprising a agent, such as an antibiotic or FU-naproxen, suspended or dispersed in a polymer, is screwed into a bone in the same manner as a prior art screw. The screw according to the present invention then releases drug, in a sustained time-wise fashion, thereby conferring therapeutic benefits, such as antibiotic, anti-inflammatory, and antiviral effects, to the tissue surrounding the device, such as muscle, bone, blood, etc.

A preferred property of a device incorporating the inventive formulation is its sustained release characteristic, wherein the rate of release of the drug from the device is primarily limited by the rate of dissolution of the drug from the granules into the matrix; whereas the permeability of the polymer is non-rate limiting with respect to the rate of release of the drug.

In embodiments according to the present invention wherein the device is a a device (e.g., surgical implement) into which the agent and polymer have been incorporated as a constituent part, the polymer is advantageously a solid having physical properties appropriate for the particular application of the device. For instance, where the device is a screw, stent, etc., the polymer is advantageously a rigid solid forming at least part of the surgical implement. In particular embodiments according to the present invention, such as where the system is part of a prosthetic joint, the polymer advantageously is non-bioerodible and remains in place after the agent has been released into the surrounding tissue. In other embodiments according to the present invention, the polymer bioerodes after release of substantially all the agent.

In exemplary embodiments, a system comprising the invention may be used to treat restenosis and related complications following percutaneous transluminal coronary angioplasty. In certain embodiments, it is important to note that the local delivery of drug/drug combinations may be utilized to treat a wide variety of conditions utilizing any number of medical devices, or to enhance the function and/or life of the device. For example, intraocular lenses placed to restore vision after cataract surgery are often compromised by the formation of a secondary cataract. The latter is often a result of cellular overgrowth on the lens surface and can be potentially minimized by combining a drug or drugs with the device. Other medical devices which often fail due to tissue in-growth or accumulation of proteinaceous material in, on and around the device, such as shunts for hydrocephalus, dialysis grafts, colostomy bag attachment devices, ear drainage tubes, leads for pace makers and implantable defibrillators can also benefit from the device-drug combination approach.

Devices which serve to improve the structure and function of tissue or organ may also show benefits when combined with the appropriate agent(s). For example, improved osteointegration of orthopedic devices to enhance stabilization of the implanted device could potentially be achieved by combining it with an agent such as a bone morphogenic protein. Similarly other medical or surgical devices, staples, anastomosis devices, vertebral disks, bone pins, hemostatic barriers, clamps, screws, plates, clips, vascular implants, tissue adhesives and sealants, tissue scaffolds, various types of dressings, bone substitutes, intraluminal devices, and vascular supports could also provide enhanced patient benefit using this drug-device combination approach.

Devices can be used to deliver such pharmaceutical agents as: antiproliferative/antimitotic agents including natural products such as vinca alkaloids (e.g., vinblastine, vincristine, and vinorelbine), paclitaxel, epidipodophyllotoxins (e.g., etoposide, teniposide), antibiotics (e.g., dactinomycin (actinomycin D) daunorubicin, doxorubicin and idarubicin), anthracyclines, mitoxantrone, bleomycins, plicamycin (mithramycin) and mitomycin, enzymes (e.g., L-asparaginase which systemically metabolizes L-asparagine and deprives cells which do not have the capacity to synthesize their own asparagine); antiplatelet agents; antiproliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., BCNU and analogs, streptozocin), trazenes-dacarbazinine (DTIC); antiproliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine (e.g., cladribine)); platinum coordination complexes (e.g., cisplatin, carboplatin), procarbazine, hydroxyurea, mitotane, aminoglutethimide; hormones (e.g., estrogen); anticoagulants (e.g., heparin, synthetic heparin salts and other inhibitors of thrombin); fibrinolytic agents (e.g., tissue plasminogen activator, streptokinase and urokinase), aspirin, dipyridamole, ticlopidine, clopidogrel, abciximab; antimigratory; antisecretory (breveldin); anti-inflammatory agents: such as adrenocortical steroids (e.g., cortisol, cortisone, fludrocortisone, prednisone, prednisolone, 6U-methylprednisolone, triamcinolone, betamethasone, and dexamethasone), non-steroidal agents (e.g., salicylic acid derivatives, e.g., aspirin; para-aminophenol derivatives, e.g., acetaminophen; indole and indene acetic acids (e.g., indomethacin, sulindact and etodalac), heteroaryl acetic acids (e.g., tolmetin, diclofenac, and ketorolac), arylpropionic acids (e.g., ibuprofen and derivatives), anthranilic acids (e.g., mefenamic acid, and meclofenamic acid), enolic acids (e.g., piroxicam, tenoxicam, phenylbutazone, and oxyphenthatrazone), nabumetone, gold compounds (e.g., auranofin, aurothioglucose, gold sodium thiomalate); immunosuppressives (e.g., cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blocker; nitric oxide donors; anti-sense oligionucleotides and combinations thereof; cell cycle inhibitors, mTOR inhibitors, and growth factor signal transduction kinase inhibitors.

In certain embodiments, the agent is formed using an opiod. Exemplary opioids include morophine derivatives, such as apomorphine, buprenorphine, codeine, dihydrocodeine, dihydroetorphine, diprenorphine, etorphine, hydrocodone, hydromorphone, levorphanol, meperidine, metopon, o-methylnaltrexone, morphine, naloxone, naltrexone, normorphine, oxycodone, and oxymorphone. In other embodiments, the opioid is a fentanyl derivative which can be derivitized to form the agent, such as β-hydroxy-3-methylfentanyl.

According to the present invention, the pharmaceutical agent may have low solubility in biological fluids, such as blood plasma, lymphatic fluid, peritoneal fluid, etc.

The present invention applies to pharmaceutically active agents. Suitable agents useful in the present invention include agents of immune response modifiers such as cyclosporin A and FK 506, corticosteroids such as dexamethasone and triamcinolone acetonide, angiostatic steroids such as trihydroxy steroids, antiparasitic agents such as atovaquone, anti-glaucoma drugs such as ethacrynic acid, antibiotics including ciprofloxacin, differentiation modulators such as retinoids (e.g., trans-retinoic acid, cis-retinoic acid and analogues), antiviral drugs including high molecular weight low (10-mers), anti-sense compounds, anticancer drugs such as BCNU, non-steroidal anti-inflammatory drugs such as indomethacin and flurbiprofen, and agents comprising a conjugate of at least two compounds linked via a reversible covalent or ionic bond that is cleaved at a desired site in a body to regenerate an active form of each compound. In some embodiments the agent is relatively insoluble in aqueous media, including physiological fluids, such as blood serum, mucous, peritoneal fluid, limbic fluid, etc. In still further embodiments, suitable agents include drugs which are lipophilic derivatives of hydrophilic drugs that are easily converted into their hydrophilic drugs under physiologic conditions. Reference may be made to any standard pharmaceutical textbook for the procedures to obtain a suitable form of a drug. In this regard, the present invention is especially suitable for agents that heretofore have not found broad application due to their inherent low solubility, or have found only limited application in oil-based or other lipid-based delivery vehicles.

In certain embodiments, the invention provides an intraluminal medical device for implantation into a lumen of a blood vessel, in particular adjacent an intraluminal lesion such as an atherosclerotic lesion, for maintaining patency of the vessel. In particular the invention provides an elongate radially expandable tubular stent having an interior luminal surface and an opposite exterior surface extending along a longitudinal stent axis, the stent having a coating on at least a portion of the interior or exterior surface thereof. The local delivery of drug combinations from a stent preferably prevents vessel recoil and remodeling through the scaffolding action of the stent and the prevention of multiple components of neointimal hyperplasia or restenosis as well as a reduction in inflammation and thrombosis. This local administration of drugs to stented coronary arteries may also have additional therapeutic benefit.

For example, higher tissue concentrations of the drugs may be achieved utilizing local delivery, rather than systemic administration. In addition, reduced systemic toxicity may be achieved utilizing local delivery rather than systemic administration while maintaining higher tissue concentrations. Also in utilizing local delivery from a device rather than systemic administration, a single procedure may suffice with better patient compliance. An additional benefit of combination drug therapy may be to reduce the dose of each of the therapeutic agents, thereby limiting their toxicity, while still achieving a reduction in restenosis, inflammation and thrombosis. Local stent-based therapy is an exmplary means of improving the therapeutic ratio (efficacy/toxicity) of anti-restenosis, anti-inflammatory, or anti-thrombotic agents.

There are a multiplicity of different medical devices that may be utilized following percutaneous transluminal coronary angioplasty. For example, a number of different stents may be prepared according the present teachings. A stent is commonly used as a tubular structure left inside the lumen of a duct to relieve an obstruction. Commonly, stents are inserted into the lumen in a non-expanded form and are then expanded autonomously, or with the aid of a second device in situ. A typical method of expansion occurs through the use of a catheter-mounted angioplasty balloon which is inflated within the stenosed vessel or body passageway in order to shear and disrupt the obstructions associated with the wall components of the vessel and to obtain an enlarged lumen.

Stents may be fabricated utilizing any number of methods. For example, a stent may be fabricated from a hollow or formed stainless steel tube that may be machined using lasers, electric discharge milling, chemical etching or other means. The stent is inserted into the body and placed at the desired site in an unexpanded form. In one exemplary embodiment, expansion may be effected in a blood vessel by a balloon catheter, where the final diameter of the stent is a function of the diameter of the balloon catheter used.

It should be appreciated that a stent in accordance with the present invention may be embodied in a shape-memory material, including, for example, an appropriate alloy of nickel and titanium or stainless steel.

Structures formed from stainless steel may be made self-expanding by configuring the stainless steel in a predetermined manner, for example, by twisting it into a braided configuration. In this embodiment, after the stent has been formed it may be compressed so as to occupy a space sufficiently small as to permit its insertion in a blood vessel or other tissue by insertion means, wherein the insertion means include a suitable catheter, or flexible rod.

On emerging from the catheter, a stent may be configured to expand into the desired configuration where the expansion is automatic or triggered by a change in pressure, temperature or electrical stimulation.

Regardless of the design of a stent, it is preferable to have the drug combination dosage applied with enough specificity and a sufficient concentration to provide an effective dosage in the lesion area. In this regard, the “reservoir size” in the coating is preferably sized to adequately apply the drug combination dosage at the desired location and in the desired amount.

In an alternate exemplary embodiment, the entire inner and outer surface of a stent may be coated with drug/drug combinations in therapeutic dosage amounts. It is, however, important to note that the coating techniques may vary depending on the drug combinations. Also, the coating techniques may vary depending on the material comprising the stent or other intraluminal medical device.

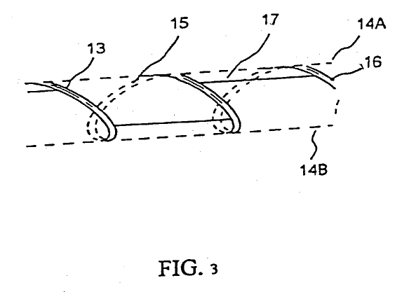

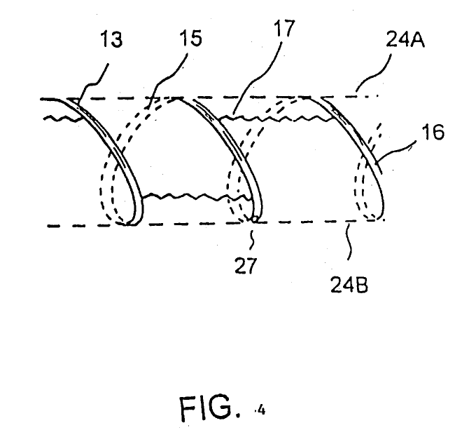

An embodiment of an intraluminal device (e.g., a stent) according to the present invention is depicted in FIGS. 3 and 4.

FIG. 3 shows a side plan view of a preferred elongate radially expandable tubular stent 13 having a surface coated with a sustained release drug delivery system in a non-deployed state. As shown in FIG. 3, the stent 13 has its radially outer boundaries 14A, 14B at a non-deployed state. The interior luminal surface 15, the exterior surface 16, or an entire surface of the stent 13 may be coated with a sustained release drug delivery system or comprise a sustained release drug delivery system. The interior luminal surface 15 is to contact a body fluid, such as blood in a vascular stenting procedure, while the exterior surface 16 is to contact tissue when the stent 13 is deployed to support and enlarge the biological vessel or duct.

In an alternate embodiment, an optional reinforcing wire 17 that connects two or more of the adjacent members or loops of the stent structure 13 is used to lock-in and/or maintain the stent at its expanded state when a stent is deployed. This reinforcing wire 17 may be made of a Nitinol or other high-strength material. A Nitinol device is well known to have a preshape and a transition temperature for said Nitinol device to revert to its preshape. One method for treating an intraluminal tissue of a patient using a surface coated stent 13 of the present invention comprises collapsing the radially expandable tubular stent and retracting the collapsed stent from a body of a patient. The operation for collapsing a radially expandable tubular stent may be accomplished by elevating the temperature so that the reinforcing wire 17 is reversed to its straightened state or other appropriate state to cause the stent 13 to collapse for removing said stent from the body of a patient.

FIG. 4 shows an overall view of an elongate radially expandable tubular stent 13 having a sustained release drug delivery system coated stent surface at a deployed state. As shown in FIG. 4, the stent 13 has its radially outer boundaries 24A, 24B at a deployed state. The interior luminal surface 14, the exterior surface 16, or an entire surface of the stent 13 may be coated or may comprise the sustained release drug delivery system. The interior luminal surface 15 is to contact a body fluid, such as blood in a vascular stenting procedure, while the exterior surface 16 is to contact tissue when the stent 13 is deployed to support and enlarge the biological vessel. The reinforcing wire 17 may be used to maintain the expanded stent at its expanded state as a permanent stent or as a temporary stent. In the case of the surface coated stent 13 functioning as a temporary stent, the reinforcing wire 17 may have the capability to cause collapsing of the expanded stent.

The deployment of a stent can be accomplished by a balloon on a delivery catheter or by self-expanding after a pre-stressed stent is released from a delivery catheter. Delivery catheters and methods for deployment of stents are well known to one who is skilled in the art. The expandable stent 13 may be a self-expandable stent, a balloon-expandable stent, or an expandable-retractable stent. The expandable stent may be made of memory coil, mesh material, and the like.

III. Other Examples

Agent TC-112 comprising a conjugate of 5-FU1 and naproxen linked via a reversible covalent bond, and agent G.531.1 comprising a conjugate of 5-FU and FA were prepared in accordance with the methods set forth in U.S. Pat. No. 6,051,576. The structure of these compounds is reproduced below.

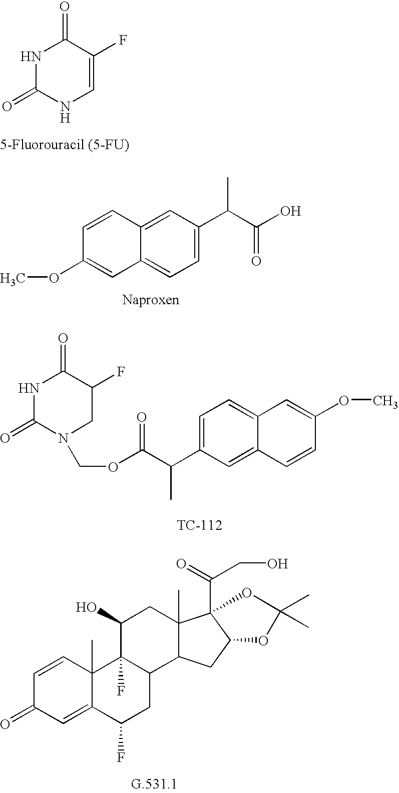

The following examples are intended to be illustrative of the disclosed invention. The examples are non-limiting, and the skilled artisan will recognize that other embodiments are within the scope of the disclosed invention.

### EXAMPLE 1

To 20 gm of 10% (w/v) aqueous poly(vinyl alcohol) (PVA) solution, 80.5 mg of agent TC-112 was dispersed. 5 pieces of glass plates were then dipping coated with this TC-112/PVA suspension and followed by air-drying. The coating and air-drying was repeated four more times. At the end about 100 mg of TC-112/PVA was coated on each glass plates. The coated glass plates were then heat treated at 135° C. for 5 hours. After cooling to room temperature, the glass plates were individually placed in 20 ml of 0.1 M mol phosphate buffer (pH 7.4, 37° C.) for release test. Sample was taken daily and entire release media were replaced with fresh one at each sampling time. The drugs and TC-112 released in the media were determined by reverse-phase HPLC. The half-life for TC-112 in pH 7.4 buffer is 456 min, in serum is 14 min.

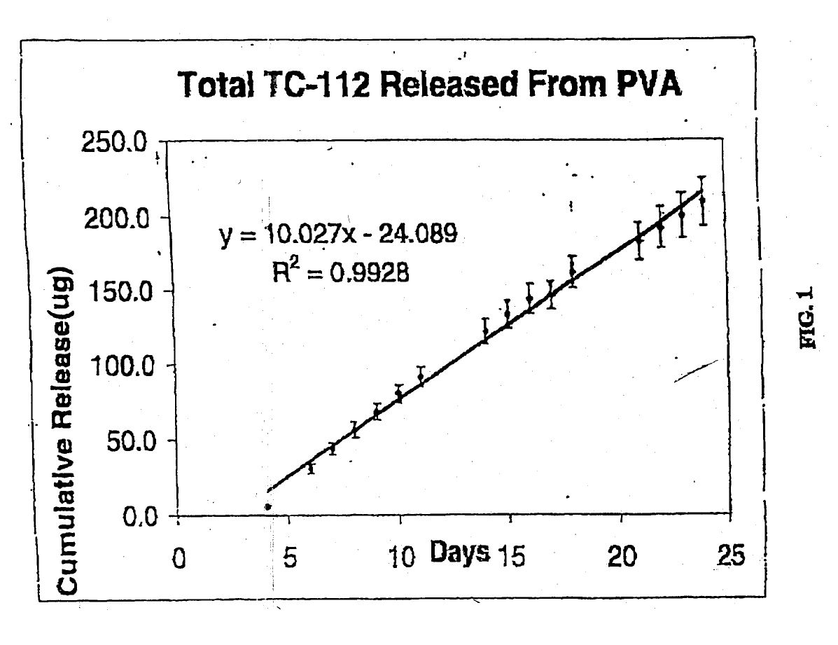

The results are shown in FIG. 1, which shows the total cumulative release of TC-112 from PVA coated glass plates. The slope of the curve demonstrates that TC-112 is released at 10 μg/day. The data represent both intact and constituents of the compound TC-112.

### EXAMPLE 2

12.0 gm of silicone part A (Med-6810A) were mixed with 1.2 gm of silicone part B (Med-6810B), and degassed in sonicator for 10 min, followed by water aspirator. 41.2 mg of (TC-112) were dispersed in this degassed silicone, and degassed again. 0.2 gm of the mixture was spread on one surface of a glass plate. The glass plates (total 5) were then placed in oven and heated at 105° C. for 20 min. to cure. After removing from the oven and cooled to room temperature, 0.2 gm of the mixture was spread on the other uncoated surface of each glass plate. The coated glass plates were then heat treated again at 105° C. for 20 min. After cooling to room temperature, the glass plates were individually placed in 20 ml of 0.1 M phosphate buffer (pH 7.4, 37° C.) for release test. Samples were taken daily, and the entire release media was replaced with fresh media at each sampling time. The drugs (5FU and TA) and TC-112 released in the media were determined by HPLC.

The total TC-112 release for silicone coating was calculated as follows. The molecular weight of Naproxen is 230.3, and the molecular weight for 5-Fluorouracil is 130.1, while the inventive compound (TC-112) generated from these two drugs has a molecular weight of 372.4. To detect x mg of naproxen, this means that x*372.4/230.3 mg of TC-112 was hydrolyzed. The total TC-112 released equals the sum of TC-112 detected in the release media and the TC-112 hydrolyzed. For example, up to day 6, 43.9 mg of naproxen is detected, this means 71.0 (43.9*372.4/230.3) mg of TC-112 was hydrolyzed, at the same time, 51.4 mg of TC-112 is detected in buffer, therefore a total of 122.4mg (51.4 plus 71.0) of TC-112 is released up to day 6.

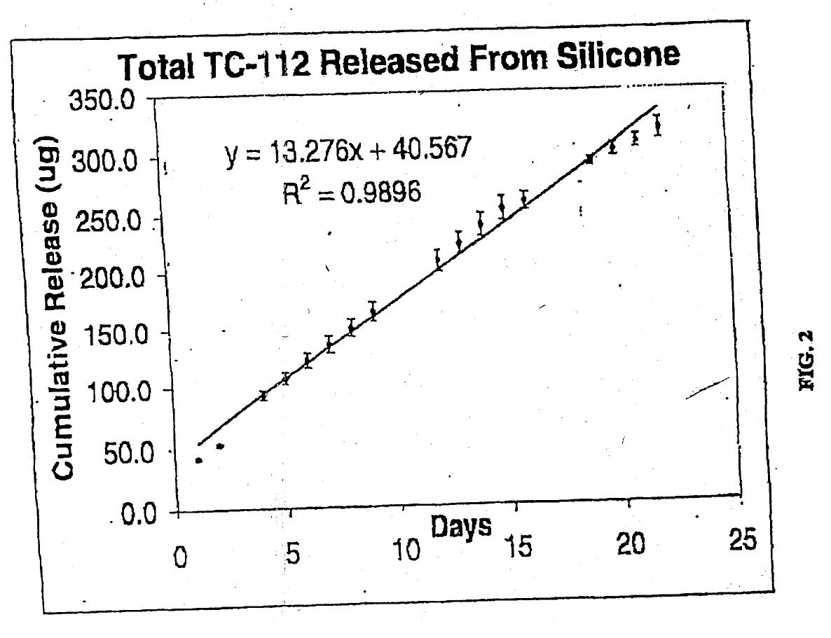

The results are shown in FIG. 2, which shows the total cumulative release of TC-112 from silicone coated glass plates. The slope of the curve demonstrates that TC-112 is released at 13.3 μg/day. Again, the data represent both intact and constituents of the inventive compound. The similarity in the slopes demonstrates that the polymers have little effect on the release of the drug.

1. A sustained-release formulation comprising:
at least one granule comprising a therapeutically effective amount of at least one agent, and
a polymer matrix coating the at least one agent, wherein the at least one agent has a rate of release from the formulation that is limited primarily by the rate at which the at least one agent dissolves into the matrix.

2. The sustained-release formulation of claim 1, wherein the at least one agent has a solubility in the polymer matrix of about 10 mg/ml or less.

3. The sustained-release formulation of claim 1, wherein the at least one agent has a solubility in the polymer matrix of about 1 mg/ml or less.

4. The sustained-release formulation of claim 1, wherein the at least one agent has a solubility in the polymer matrix of about 0.1 mg/ml or less.

5. The sustained-release formulation of claim 1, wherein the at least one agent has a solubility in the polymer matrix of about 0.01 mg/ml or less.

6. The sustained-release formulation of claim 1, wherein sustained release of the at least one agent occurs for a period of at least 3 hours.

7. The sustained-release formulation of claim 1, wherein diffusion of the at least one agent through the polymer matrix is primarily non-release-rate-limiting with respect to the rate of release of the at least one agent from the matrix.

8. The sustained-release formulation of claim 1, wherein the polymer matrix is a hydrogel.

9. The sustained-release formulation of claim 1, wherein the at least one agent is a codrug.

10. The sustained-release formulation of claim 1, wherein the polymer matrix is a biocompatible fluid or semisolid, in either case selected so that the at least one agent has low solubility therein.

11. The sustained-release formulation of claim 10, wherein the semisolid contains long chain polyethylene glycol (PEG).

12. The sustained release formulation of claim 1, wherein the microenvironment of the polymer matrix has a non-physiological pH.

13. The sustained-release formulation of claim 12, wherein the microenvironment of the polymer matrix has a neutral pH.

14. The sustained-release formulation of claim 1, wherein the at least one agent has low solubility in water.

15. The sustained-release formulation of claim 1, wherein the at least one agent has moderate solubility in water.

16. The sustained-release formulation of claim 1, wherein the at least one agent is not ionized within the polymer matrix.

17. The sustained-release formulation of claim 1, wherein the polymer matrix is non-bioerodible.

18. The sustained-release formulation of claim 1, wherein the polymer matrix is bioerodible.

19. The sustained-release formulation of claim 1, wherein the polymer matrix is impermeable to peptides or proteins of about 10 kD or greater.

20. The sustained-release formulation of claim 1, further comprising a bio-adhesive or muco-adhesive coating covering at least a portion of said formulation.

21. The sustained-release formulation of claim 1, wherein the formulation is affixed to a physiological system.

22. A drug delivery device comprising:
a substrate having a surface, and
a sustained-release formulation adhered to the surface, the sustained-release formulation comprising a therapeutically effective amount of at least one agent, wherein the at least one agent is dispersed in granular form within a polymer matrix and has a solubility in the polymer matrix of about 0.01 mg/ml or less.

23. The device of claim 22, wherein the release rate of the at least one agent is limited primarily by the rate at which the at least one agent dissolves within the matrix.

24. The device of claim 22, wherein the polymer matrix is a hydrogel.

25. The device of claim 22, wherein the at least one agent is a codrug.

26. The device of claim 22, wherein the polymer matrix is a biocompatible fluid or semisolid, in either case selected so that the at least one agent has low solubility therein.

27. The device claim 26, wherein the semisolid is contains long chain polyethylene glycol (PEG).

28. The device of claim 22, wherein the microenvironment of the polymer matrix has a non-physiological pH.

29. The sustained-release formulation of claim 28, wherein the microenvironment of the polymer matrix has a neutral pH.

30. The device of claim 22, wherein the at least one agent is of low solubility in water.

31. The device of claim 22, wherein the at least one agent is of moderate solubility in water.

32. The device of claim 22, wherein the at least one agent is not ionized within the polymer matrix.

33. The device of claim 22, wherein the polymer matrix is non-bioerodible.

34. The device of claim 22, wherein the polymer matrix is bioerodible.

35. The device of claim 22, wherein the polymer matrix is impermeable to proteins or peptides of about 10 kD or greater.

36. The device of claim 22, further comprising a bio-adhesive or muco-adhesive coating covering at least a portion of said formulation.

37. The device of claim 22, wherein the formulation is affixed to a physiological system.

38. A method of providing sustained-release administration of granular drugs comprising:
providing a therapeutically effective amount of at least one agent in granular form;
forming a sustained-release formulation by combining the at least one agent with a polymer matrix such that the at least one agent remains substantially in granular form, wherein the at least one agent has a solubility in the polymer matrix of about 0.01 mg/ml or less; and administering the sustained-release formulation to a patient.

39. The method of claim 38, wherein the release rate of the at least one agent is limited primarily by the rate at which the at least one agent dissolves within the matrix.

40. The method of claim 38, wherein the polymer matrix is a hydrogel.

42. The method of claim 38, wherein the at least one agent is a codrug.

43. The method of claim 38, wherein the polymer matrix is a biocompatible fluid or semisolid, in either case selected so that the at least one agent has low solubility therein.

44. The method of claim 43 wherein the semisolid comprises long chain polyethylene glycol (PEG).

45. The method of claim 38 wherein the microenvironment of the polymer matrix has a non-physiological pH.

46. The method of claim 45, wherein the microenvironment of the polymer matrix has a neutral pH.

47. The method of claim 38, wherein the at least one agent has low solubility in water.

48. The method of claim 38, wherein the at least one agent has moderate solubility in water.

49. The method of claim 38, wherein the at least one agent is not ionized within the polymer matrix.

50. The method of claim 38, wherein the polymer matrix is non-bioerodible.

51. The method of claim 38, wherein the polymer matrix is bioerodible.

52. The method of claim 38, wherein the polymer matrix is impermeable to peptides or proteins of about 10 kD or greater.

53. The method of claim 38, further comprising a bio-adhesive or muco-adhesive coating covering at least a portion of said formulation.

54. The method of claim 38, wherein the formulation is affixed to a physiological system.

55. The method of claim 38 further comprising providing the sustained-release formulation in a pharmaceutically acceptable carrier.

56. A sustained-release formulation comprising:
a plurality of granules comprising a therapeutically effective amount of a codrug, and
a polymer matrix, wherein the polymer matrix is essentially non-release rate limiting with respect to the rate of release of the agent from the matrix.

57. A sustained-release formulation comprising:
a polymer matrix surrounded by physiological tissue, and
a plurality of granules comprising a therapeutically effective amount of a codrug dispersed in said matrix, wherein the granules have a surface area that is at least partially exposed to the surrounding tissue, and wherein the release rate of the codrug from the formulation is proportional to the exposed surface area of the granules.

58. A sustained-release formulation comprising:
a plurality of granules comprising a therapeutically effective amount of a codrug having a form selected from I, Ia, II, IIa, III, IIIa, and IV below,
A₁*(—L—A₂*)ₙ (I)
A₁*(—A₂*)ₙ (Ia)
A₁*—L—A₂* (II)
A₁*—A₂* (IIa)
A₂*—L—A₁*—L—A₂* (III)
A₂*—A₁*—A₂* (IIIa),
Wherein A₁*is a residue of a first biologically active compound A_{1,}
A₂*is a residue of a second biologically active compound A_{2,}
L is a linking group selected from a direct bond and a divalent organic linking group, and
n is an integer having a value of from 1 to 4; and a polymer matrix, coating the at least one agent, wherein the at least one
agent has a rate of release from the formulation that is limited primarily by the rate at which the at least one agent dissolves into the matrix.

59. The sustained-release formulation of any of claims 56, 57, or 58 wherein the codrug is in prodrug form.
